(19) United States
(12) Patent Application Publication
(10) Pub. No.: US 2025/0262199 A1
(43) Pub. Date: Aug. 21, 2025

(54) TREATMENT OF AUTOIMMUNE SKIN DISEASE

(71) Applicant: ASLAN PHARMACEUTICALS PTE LTD, Singapore (SG)

(72) Inventors: Robert MOORE, Singapore (SG); Isana ENDO, Singapore (SG); Carl FIRTH, Singapore (SG); Stephen DOYLE, Singapore (SG)

(21) Appl. No.: 18/858,483

(22) PCT Filed: Apr. 21, 2022
(86) PCT No.: PCT/SG2022/050239

§ 371 Date: Oct. 21, 2024

(51) Int. Cl. A61K 31/455; A61K 45/06; A61P 17/00; A61P 17/14; A61P 37/06

(52) U.S. Cl. CPC A61K 31/455; A61K 45/06; A61P 17/00; A61P 17/14; A61P 37/06

(57) ABSTRACT

A method of treating an autoimmune skin disease in a patient comprising administering a therapeutically effective amount of a DHODH inhibitor or a pharmaceutically acceptable salt thereof. Also provided are formulations or compositions suitable for treating autoimmune skin diseases.

The present disclosure relates to use of a DHODH inhibitor for the treatment of an autoimmune skin disease, such as vitiligo and/or alopecia, alone or in combination with another therapy.

### BACKGROUND

An organ that is commonly affected by autoimmune diseases is the skin. There are a large number of different autoimmune skin diseases, including vitiligo, alopecia, psoriasis, eczema, dermatomyositis, and scleroderma. Common symptoms of autoimmune skin diseases include itchy skin, rashes, blisters, lesions, and scaly patches. Since the skin is the largest organ and is exposed to the environment, many of these symptoms are highly visible. As a result, autoimmune skin diseases often have a substantial psychological and social impact on patients-recent studies have shown that patients with autoimmune skin diseases are more likely to have depressive symptoms, experience social isolation and have a lower quality of life.

Existing treatments for autoimmune skin diseases are typically topical medicines, such as antihistamines, corticosteroid creams, calcineurin inhibitors such as tacrolimus, and topical anaesthetics, such as capsaicin. However, in some instances, autoimmune skin diseases are not adequately controlled by topical medicines. In addition, it is not advisable for some patients to take the available topical medicines.

Accordingly, there is a need for alternative treatments in order to manage and treat autoimmune skin diseases, such as vitiligo and alopecia.

Autoimmune disease pathology is characterised the body attacking itself with aberrant T cells and/or B cells. Purine and pyrimidine nucleotides play critical roles in DNA and RNA synthesis as well as in membrane lipid biosynthesis and protein glycosylation. They are necessary for the development and survival of mature T lymphocytes. Activation of T lymphocytes is associated with an increase of purine and pyrimidine pools, which in turn leads to a marked increase in activity of key enzymes involved in de novo purine and pyrimidine synthesis.

Vitiligo (leukoderma) is loss of function or death of melanocytes (pigment cells) in skin, membranes and/or hair. The exact cause of vitiligo is unknown. It is believed to be due to genetic susceptibility that is triggered by an environmental factor such that an autoimmune disease occurs. This results in the destruction of pigment cells. Risk factors include a family history of the condition or other autoimmune diseases, such as hyperthyroidism, Alopecia areata, and pernicious anaemia.

There is no known cure for vitiligo. For those with light skin, sunscreen and makeup are all that is typically recommended. Other treatment options may include steroid creams or phototherapy to darken the light patches. Alternatively, efforts to lighten the unaffected skin, such as with hydroquinone, may be tried. Several surgical options are available for those who do not improve with other measures. A combination of treatments generally has better outcomes. Counselling to provide emotional support may be useful.

Globally about 1% of people are affected by vitiligo. In some populations it affects as many as 2-3%. Males and females are equally affected. About half show the disorder before age 20 and most develop it before age 40.

Depending on the type of vitiligo you have, it may affect:
Nearly all skin surfaces With this type, called universal vitiligo, the discoloration affects nearly all skin surfaces.
Many parts of the body. With this most common type, called generalized vitiligo, the discolored patches often progress similarly on corresponding body parts (symmetrically).
Only one side or part of the body. This type, called segmental vitiligo, tends to occur at a younger age, progress for a year or two, then stop.
One or only a few areas of the body. This type is called localized (focal) vitiligo.
The face and hands. With this type, called acrofacial vitiligo, the affected skin is on the face and hands, and around body openings, such as the eyes, nose and ears.

It's difficult to predict how your disease will progress. Sometimes the patches stop forming without treatment. In most cases, pigment loss spreads and eventually involves most of the skin. Occasionally, the skin gets its colour back.

Vitiligo is sometimes associated with autoimmune and inflammatory diseases, such as Hashimoto's thyroiditis scleroderma, rheumatoid arthritis, type 1 diabetes mellitus, psoriasis, Addison's disease, pernicious anaemia, Alopecia areata, systemic lupus erythematosus, and celiac disease.

Among the inflammatory products of NALP1 are caspase 1 and caspase 7, which activate the inflammatory cytokine interleukin-1β. Interleukin-1β and interleukin-18 are expressed at high levels in people with vitiligo.

In one of the mutations, the amino acid leucine in the NALP1 protein was replaced by histidine (Leu155→His). The original protein and sequence is highly conserved in evolution, and is found in humans, chimpanzee, rhesus monkey, and the bush baby. Addison's disease (typically an autoimmune destruction of the adrenal glands) may also be seen in individuals with vitiligo.

Whilst not wishing to be bound by theory, it is believed that in one embodiment the present treatment is disease modifying, for example data presented in the Examples suggests that the present treatment has the ability to change the status of premelanosome protein (PMEL) primarily expressed in pigment cells, in the dermis and epidermis. It suggests that the treatment can be used to restore some element of balance to the immune system. This is interesting because it works in a totally different way to current treatments. What is more, the therapy of the present disclosure may be employed in combination with existing treatments with good effect.

Alopecia areata, also known as spot baldness, is a condition in which hair is lost from some or all areas of the body. Psychological stress and illness are possible factors in bringing on Alopecia areata in individuals at risk, but in most cases, there are no obvious triggers. In addition, Alopecia areata shares genetic risk factors with other autoimmune diseases. In addition, Alopecia areata shares genetic risk factors with other autoimmune diseases, such as vitiligo.

Alopecia areata is believed to be an autoimmune disease resulting from a breach in the immune privilege of the hair follicles. Risk factors include a family history of the condition. Among identical twins, if one is affected, the other has about a 50% chance of also being affected. The underlying mechanism involves failure by the body to recognize its own cells, with subsequent immune-mediated destruction of the hair follicle

No cure for the condition is known. Efforts may be used to try to speed hair regrowth, such as cortisone injections. About 0.15% of people are affected at any one time, and 2% of people are affected at some point in time. Onset is usually in childhood. Females are affected at higher rates than males.

Thus, Alopecia areata is thought to be a systemic autoimmune disorder in which the body attacks its own anagen hair follicles and suppresses or stops hair growth. For example, T cell lymphocytes cluster around affected follicles, causing inflammation and subsequent hair loss. Hair follicles in a normal state are thought to be kept secure from the immune system, a phenomenon called immune privilege. A breach in this immune privilege state is considered as the cause of Alopecia areata.

In cases of severe hair loss, limited success has been achieved by using the corticosteroid medications clobetasol or fluocinonide, corticosteroid injections, or cream. Application of corticosteroid creams to the affected skin is less effective and takes longer to produce results. Steroid injections are commonly used in sites where the areas of hair loss on the head are small or especially where eyebrow hair has been lost. Whether they are effective is uncertain. Some other medications that have been used are minoxidil, Elocon (mometasone) ointment (steroid cream), irritants (anthralin or topical coal tar), and topical immunotherapy ciclosporin, sometimes in different combinations. Topical corticosteroids frequently fail to enter the skin deeply enough to affect the hair bulbs, which are the treatment target, and small lesions typically also regrow spontaneously. Oral corticosteroids may decrease the hair loss, but only for the period during which they are taken, and these medications can cause serious side effects. No one treatment is effective in all cases, and some individuals may show no response to any treatment. Few treatments have been well evaluated. A 2008 meta-analysis of oral and topical corticosteroids, topical ciclosporin, photodynamic therapy, and topical minoxidil showed no benefit of hair growth compared with placebo, especially with regard to long-term benefits. For more severe cases, recent studies have shown promising results with the individual use of the immunosuppressant Methotrexate or adjunct use with corticosteroids. However, as relapse of the condition may occur long-term treatment is recommended.

None of the existing therapeutic options are curative or preventive. Additional therapies that work by different mechanisms would be useful.

The present inventors have shown that the fraudostat is able to influence hair growth. What is more it is able to address aberrant T cells, which are thought to be the underlying cause of the alopecia.

Pyrimidine is believed to be important for controlling progression from early to intermediate S phase of T cell life cycle. Inhibition of pyrimidine also causes apoptosis of activated T cells.

Similarly, biosynthesis of pyrimidine is also important in the life cycle of activated B cells.

Whilst not wishing to be bound by theory the present inventors believe that blockade of the biosynthesis of pyrimidine causes cell cycle arrest and/or apoptosis in activated T cells and/or B cells with aberrant activity in autoimmune disease.

Dihydroorotate dehydrogenase (DHODH) is the enzyme that catalyzes the fourth step in the pyrimidine biosynthetic pathway namely the conversion of dihydroorotate to orotate concomitantly with an electron transfer to ubiquinone (cofactor Q) via a flavin mononucleotide intermediate (Loffler Mol Cell Biochem, 1997). In contrast to parasites (Plasmodium falciparum) (McRobert et al Mol Biochem Parasitol 2002) and bacteria (E. coli) which exclusively have this de novo pathway as the source of pyrimidines, mammal cells have an additional salvage pathway.

During homeostatic proliferation the salvage pathway, which is independent of DHODH, seems sufficient for the cellular supply with pyrimidine bases. However, in cells with a high turnover the de novo pathway is required to proliferate. In these cells, DHODH inhibition stops the cell cycle progression by suppressing DNA synthesis and ultimately cell proliferation (Breedveld F. C. Ann Rheum Dis 2000).

There are some suggestions that inhibition of mitochondrial cytochrome bc1, a component of the electron transport chain complex III, leads to activation of p53, followed by apoptosis induction. The mitochondrial respiratory chain is coupled to the de novo pyrimidine biosynthesis pathway via the mitochondrial enzyme dihydroorotate dehydrogenase (DHODH).

The drug profile of each DHODH inhibitor is very different, for example side effects of leflunomide include arterial hypertension, myelosuppression, nausea and hair loss. Brequinar is generally employed only as a model compound because clinical trials suggest the molecule lacks the requisite activity in vivo. Vidofludimus is a next generation DHODH inhibitor, which inhibits production of proinflammatory cytokines (such as IL-17) from activated lymphocytes. However, the latter is thought to be independent of DHODH activity.

Whilst DHODH inhibitors have been of interest as therapeutics, it has been difficult to find molecules that balance all the requisite criteria, to provide a therapeutic that is effective in vivo. The present disclosure provides use of a specific DHODH inhibitor farudostat (previously known as ASLAN003) in the treatment of an autoimmune skin disease, such vitiligo and/or alopecia.

Farudostat has activity against the underlying cause of autoimmune skin diseases, namely aberrant T cells and/or B cells, which surprisingly translates in vivo to the broad-spectrum activity against autoimmune skin diseases wherein the off-target effects are minimal, in particular liver toxicity.

Farudostat has high affinity for DHODH and is effective in the clinic. Farudostat is a next generation DHODH inhibitor which is well tolerated and delivers excellent results to patients. It has the ability to positively impact on patient quality of life to control disease status, and to halt its progression and/or put the disease into remission. Advantageously, healthy cells which have a lower metabolic burden are generally unaffected by the treatment. The balanced characteristics of the treatment are extremely beneficial to patients.

That is after a period of treatment the body is able to reset itself and send the autoimmune disease into remission, for example without the need to continue administering the therapy or where the therapy is continued at a low dose for maintenance.

### SUMMARY OF THE DISCLOSURE

The present disclosure is summarised in the following paragraphs:

1. A method of treating an autoimmune skin disease in a patient comprising administering a therapeutically effective amount of a DHODH inhibitor 2-(3,5-difluoro-3′methoxybiphenyl-4-ylamino) nicotinic acid or a pharmaceutically acceptable salt thereof.

1A A DHODH inhibitor 2-(3,5-difluoro-3′methoxybiphenyl-4-ylamino) nicotinic acid or a pharmaceutically acceptable salt thereof for use in a patient with or suspected of having an autoimmune skin disease.

1B Use of a DHODH inhibitor 2-(3,5-difluoro-3′methoxybiphenyl-4-ylamino) nicotinic acid or a pharmaceutically acceptable salt thereof in the manufacture of a medicament for the treatment of an autoimmune skin disease.

2. A method, inhibitor or use according to any one of the preceding paragraphs, wherein the autoimmune skin disease is characterised by aberrant T cell responses.

3. A method, inhibitor or use according to any one of the preceding paragraphs, wherein the autoimmune skin disease is characterised by aberrant B cell responses.

4. A method, inhibitor or use according to any one of the preceding paragraphs, wherein the autoimmune skin disease is inadequately controlled, for example by standard of care medicine.

5. A method, inhibitor or use according to any one of the preceding paragraphs, wherein the autoimmune skin disease is severe.

6. A method, inhibitor or use according to any one of any one of the preceding paragraphs, wherein the autoimmune skin disease is selected from the group comprising: vitiligo, alopecia, atopic dermatitis, Behcet's disease, dermatitis herpetiformis, dermatomyositis, lichen planus, linear IgA disease, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, and vesiculobullous dermatosis.

7. A method, inhibitor or use according to any one of any one of the preceding paragraphs, wherein the autoimmune skin disease is selected from halo nevus, idiopathic guttate hypomelanosis, peibaldsim, pityriasis and progressive macular hypomelanosis.

8. A method, inhibitor or use according to any one of the preceding paragraphs, wherein the autoimmune skin disease is selected from the group comprising vitiligo and alopecia.

9. A method, inhibitor or use according to any one of the preceding paragraphs, wherein the autoimmune skin disease is vitiligo, such as segmental or non-segmental vitiligo.

10. A method, inhibitor or use according to any one of the preceding paragraphs, wherein the autoimmune skin disease is alopecia, such as diffuse Alopecia areata, Alopecia areata monolocularis, ophiasis Alopecia areata, Alopecia areata barbae, Alopecia areata totallis or Alopecia areata universalis.

11. A method, inhibitor or use according to any one of the preceding paragraphs, wherein the autoimmune skin disease is vitiligo and alopecia.

12. A method, inhibitor or use according to any one of paragraphs 1 to 5, wherein the autoimmune skin disorder is not one or more of the following: atopic dermatitis, Behcet's disease, dermatitis herpetiformis, dermatomyositis, lichen planus, linear IgA disease, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, or vesiculobullous dermatosis.

13. A method, inhibitor or use according to any one of the preceding paragraphs, wherein frequency or severity of relapses are reduced, with treatment.

14. A method, inhibitor or use according to any one of the preceding paragraphs, wherein aberrant T cell activation is minimised, for example progression from early to intermediate S phase of T cell life cycle is inhibited.

15. A method, inhibitor or use according to paragraph 13, wherein there is decreased activation of memory T cells.

16. A method, inhibitor or use according to paragraph 13 or 14, wherein aberrant activation of TH1 T cells is inhibited.

17. A method according to any one of the preceding paragraphs, wherein TH2 T cell activation is not inhibited.

18. A method, inhibitor or use according to any one of the preceding paragraphs, wherein aberrant B cell activation is inhibited, for example through limiting progression of S phase in the cell life cycle is inhibited.

19. A method, inhibitor or use according to any one of the preceding paragraphs, wherein apoptosis of aberrant immune cells in stimulated.

20. A method, inhibitor or use according to any one of the preceding paragraphs, wherein the treatment is for a flare-up of the autoimmune disease.

21. A method, inhibitor or use according to any one of the preceding paragraphs, wherein the treatment is continuous, for example 100 to 400 mg per dose (such as per day), in particular 300 to 400 mg per dose (such as per day) for at least a period and optionally a maintenance for example 100 to 200 mg per dose (such as per day).

22. A method, inhibitor or use according to any one of the preceding paragraphs, wherein the treatment is intermittent or is discontinued after a period, for example after a defined endpoint.

23. A method, inhibitor or use according to any one of the preceding paragraphs, wherein the DHODH inhibitor is employed as monotherapy.

24. A method, inhibitor or use according to any one of paragraphs 1 to 21, wherein the DHODH inhibitor is employed in a combination therapy.

25. A method, inhibitor or use according to paragraph 23, wherein the combination therapy comprises a treatment independently selected from corticosteroids (for example oral prednisone and intravenous methylprednisolone), plasma exchange (plasmapheresis), interferon beta medications, glatiramer acetate, fingolimod, dimethyl fumarate, diroximel fumarate, teriflunomide, siponimod, cladribine, ocrelizumab, natalizumab, an anti-CD20 agent or biosimilar thereof, such as rituximab, alemtuzumab, and a Bruton's Tyrosine Kinase (BTK) inhibitor.

26. A method, inhibitor or use according to any one of paragraphs 23 to 24, wherein the combination therapy comprises an antidepressant, for example a tricyclic antidepressant, such as clomipramine.

27. A method, inhibitor or use according to any one of paragraphs 23 to 25, wherein the combination therapy comprises duloxetine.

28. A method, inhibitor or use according to any one of paragraphs 23 to 26, wherein the combination comprises mirabegron and/or desmopressin.

29. A method, inhibitor or use according to any one of paragraphs 23 to 27, wherein the combination therapy comprises an inhibitor of interferon gamma (IFN-γ) signalling, such as an IFN-γ inhibitor (for example dexamethasone or an anti-IFN-γ antibody) or a JAK inhibitor (for example an anti-JAK antibody such as ruxolitinib).

30. A method, inhibitor or use according to any one of paragraphs 23 to 28, wherein the combination therapy comprises an anti-CD20 agent or a biosimilar thereof, for example Rituxan (rituximab), a Rituximab biosimilar, Gazyva, Kesimpta, Ocrevus (ocrelizumab), Ruxience, Truxima, Zevalin, Arzerra, AcellBia, HLX01, Reditux, Ritucad or Zytux.

31. A method, inhibitor or use according to any one of paragraphs 29 to 29, wherein the combination therapy comprises a Bruton's Tyrosine Kinase (BTK) inhibitor, for example Ibrutinib, Acalabrutinib, Zanubrutinib, Evobrutinib, ABBV-105, Fenebrutinib, GS-4059, Spebrutinib or HM71224.

32. A method, inhibitor or use according to any one of paragraphs 23 to 30, wherein the combination comprises methotrexate or does not comprise methotrexate.

33. A method, inhibitor or use according to any one of paragraphs 23 to 31, wherein the combination comprises a purine synthesis inhibitor, such as azathioprine.

34. A method, inhibitor or use according to any one of paragraphs 23 to 33, wherein the combination therapy comprises an antihistamine.

35. A method, inhibitor or use according to any one of paragraphs 23 to 34, wherein the combination therapy is a corticosteroid, for example an oral steroid, cortisone (injection such as local injection) and/or corticosteroid cream (for example mometasone, clobetasol, cortisone, fluocinonide)

36. A method, inhibitor or use according to any one of paragraphs 23 to 35, wherein combination therapy is minoxidil.

37. A method, inhibitor or use according to any one of paragraphs 23 to 36, wherein the combination therapy is anthralin or topical coal tar.

38. A method, inhibitor or use according to any one of paragraphs 23 to 37, wherein the combination therapy is an immunosuppressant, for example ciclosporin and/or tacrolimus.

39. A method, inhibitor or use according to any one of paragraph 23 to 38, wherein the combination therapy is phototherapy, for example UVB.

40. A method, inhibitor or use according to any one of paragraphs 29 to 39, wherein the combination comprises a biological therapeutic such as an antibody or binding fragment thereof in particular dupilumab or an anti-IL-13Rx1 antibody or antigen binding fragment thereof, such as ASLAN004, including a pharmaceutical formulation of any one of the same.

41. A method, inhibitor or use according to any one of the preceding paragraphs, wherein the DHODH inhibitor is administered at a dose in the range 1 mg to 400 mg per day, for example 100 mg to 400 mg per day, such as 100, 200, 300 or 400 mg per day.

42. A method, inhibitor or use according to any one of the preceding paragraphs, wherein the DHODH inhibitor is administered daily, for example once daily or twice daily.

43. A method, inhibitor or use according to any one of the preceding paragraphs, wherein the patient is human.

44. A method, inhibitor or use according to any one of the preceding paragraphs 1, wherein the patient has an age of at least 40 years, for example at 45, at least 50, at least 55, at least 60, at least 65, at least 70, at least 75.

45. A method, inhibitor or use according to any one of the preceding paragraphs, wherein the patient has co-morbidities.

46. A method, inhibitor or use according to paragraph 38, wherein the co-morbidity is selected from obesity, allergy, asthma, COPD, diabetes, kidney failure, heart disease (including heart failure), cancer, dementia, liver disease, and combinations thereof.

In one embodiment the dose is tailored to the patient.

Surprisingly, the present inventors have established that the symptoms and severity of autoimmune skin diseases such as vitiligo and/or alopecia can be treated and/or ameliorated by administering farudostat to subjects in need thereof.

In one embodiment the autoimmune skin disease is selected from the group comprising: vitiligo, alopecia, Behcet's disease, dermatitis herpetiformis, dermatomyositis, lichen planus, linear IgA dermatosis, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, vesiculobullous dermatosis and atopic dermatitis.

In one embodiment the autoimmune skin disease is not atopic dermatitis (including autoimmune progesterone dermatitis), psoriasis, erythema (including erythema nodosum), scleroderma or lupus.

In one embodiment the autoimmune skin disease is not atopic dermatitis.

In one embodiment the autoimmune skin disease is not psoriasis.

In one embodiment the autoimmune skin disease is not erythema.

In one embodiment the autoimmune skin disease is not scleroderma.

In one embodiment the autoimmune skin disease is not lupus.

In one embodiment the autoimmune skin disease is not Behcet's disease.

In one embodiment the autoimmune skin disease is not bullous pemphigoid.

In one embodiment the autoimmune skin disease is not IgA pemphigus.

In one embodiment the autoimmune skin disease is not pemphigoid.

In one embodiment the autoimmune skin disease is not ocular cicatricial pemphigoid.

In one embodiment the autoimmune skin disease is not pemphigoid gestationis (PG), pemphigus vulgaris (PV), or pemphigus folliaceus (PF).

In one embodiment the autoimmune skin disease is not vesiculobullous dermatosis.

In one embodiment the autoimmune skin disease is not dermatosmyositis.

In one embodiment the autoimmune skin disease is not lichen planus.

In one embodiment the autoimmune skin disease is not dermatitis herpetiformis.

In one embodiment the autoimmune skin disease is not vesiculobullous dermatosis.

In one embodiment the autoimmune skin disease is not linear IgA dermatosis.

In one embodiment the autoimmune skin disease is not vasculitis.

In one embodiment the autoimmune skin disease is selected from the group comprising vitiligo and alopecia.

In one embodiment the autoimmune skin disease is vitiligo and alopecia.

In one embodiment the autoimmune skin disease is vitiligo.

In one embodiment the autoimmune skin disease is vitiligo is selected from the group comprising segmental vitiligo or non-segmental vitiligo.

In one embodiment the vitiligo is segmental vitiligo.

In one embodiment the vitiligo is non-segmental vitiligo (for example focal vitiligo, generalised vitiligo or acrofacial vitiligo).

In one embodiment the autoimmune skin disease is alopecia.

In one embodiment the alopecia is selected from the group comprising diffuse Alopecia areata, Alopecia areata monolocularis, ophiasis Alopecia areata, Alopecia areata barbae, Alopecia areata totallis or Alopecia areata universalis.

In one embodiment the alopecia is diffuse Alopecia areata.

In one embodiment the alopecia is Alopecia areata monolocularis.

In one embodiment the alopecia is ophiasis Alopecia areata.

In one embodiment the alopecia is Alopecia areata barbae.

In one embodiment the alopecia is Alopecia areata totallis.

In one embodiment the alopecia is Alopecia areata universalis.

### BRIEF SUMMARY OF THE FIGURES

FIG. 1 graph showing mean vitiligo score for viltiligo mouse model after treatment with vehicle, 75 mg/kg/day farudostat, 150 mg/kg/day farudostat or anti-IFNγ

FIG. 2A graph showing mean PMEL T cell counts from skin draining lymph nodes of viltiligo mouse model after treatment with vehicle, 75 mg/kg/day farudostat, 150 mg/kg/day farudostat or anti-IFNγ

FIG. 2B graph showing mean PMEL T cell counts from dermis of viltiligo mouse model after treatment with vehicle, 75 mg/kg/day farudostat, 150 mg/kg/day farudostat or anti-IFNγ

FIG. 2C graph showing mean PMEL T cell counts from epidermis of viltiligo mouse model after treatment with vehicle, 75 mg/kg/day farudostat, 150 mg/kg/day farudostat or anti-IFNγ

FIG. 2D graph showing mean PMEL T cell counts from spleen of viltiligo mouse model after treatment with vehicle, 75 mg/kg/day farudostat, 150 mg/kg/day farudostat or anti-IFNγ

FIG. 3 graph showing mean % hair coverage of Alopecia areata mouse model after treatment with vehicle, 150 mg/kg/day farudostat, or 300 mg/kg/day farudostat

### DETAILED DESCRIPTION

2-(3,5-difluoro-3′-methoxybiphenyl-4-ylamino) nicotinic acid (referred to herein as Farudostat or ASLAN003) has the structure:

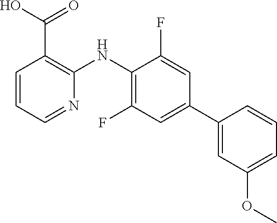 [SMILES: COc1cccc(-c2cc(F)c(Nc3ncccc3C(=O)O)c(F)c2)c1]

### Autoimmune Disease

Autoimmune disease as used herein refers to any disease or condition wherein an individual's immune system mistakenly targets that individual's own normal “healthy” cells, in particular characterised by aberrant T cell and/or B cell activation.

Aberrant T cell and/or B cell activation as employed herein refers to abnormal T cell and/or B cell activation, in particular where the abnormal cells recognise self or self antigens.

Severe autoimmune disease is where the disease is not controlled by standard of care medicaments/treatments.

Flare, is a period of disease exacerbation.

Autoimmune skin disease or disorder as used herein refers to any disease or condition wherein an individual's immune system, for example the individual's T cells and/B cells, mistakenly targets that individual's skin tissue or cells therein, including hair follicles. Farudostat is able to block the biosynthesis of pyrimidine, which is thought to cause cell cycle arrest leading to a reduction in pro-inflammatory cytokine production and/or apoptosis in T cells and/or B cells. Thus, farudostat is believed to have the potential to treat a variety of autoimmune skin diseases, such as vitiligo and alopecia.

Vitiligo as used herein refers to an autoimmune skin disease characterised by the loss of patches of skin pigmentation. Vitiligo occurs when melanocytes die or stop functioning. This results in a loss of melanin which is responsible for skin pigmentation. The pale areas of skin are more vulnerable to sunburns, making it important that proper precautions are taken by vitiligo patients. The condition can affect the skin on any part of the body, but more typically presents on the face, neck, hands and in skin creases. Vitiligo may also affect hair, or the inside of the mouth.

There are 2 main types of vitiligo-non-segmental and segmental vitiligo. Non-segmental vitiligo is significantly more common than segmental vitiligo, with about 90% of patients having this form of the disease. Patients with non-segmental vitiligo typically have symptoms on both side of the body, i.e. the white patches tend to present as symmetrical patches. In contrast, segmental vitiligo typically presents as white patches on only one area of the body.

There are no treatments currently available which can stop the process of vitiligo. Thus, the white patches caused by vitiligo are usually permanent. Available treatments, such as steroid creams, phototherapy or camouflage creams are therefore focussed on reducing the appearance of the white patches.

Alopecia areata, alopecia or spot baldness as used herein refers to an autoimmune skin disorder wherein the hair follicles are targeted by the immune system. This results in the suppression of hair growth and hair loss. The loss of hair typically occurs in clumps and may grow back, although the hair may fall out again. Alopecia can be further sub-classified as:
Diffuse Alopecia areata, where the hair loss is diffusely distributed over the whole scalp;
Alopecia areata monolocularis, where hair loss is only in one spot;
Alopecia areata multilocularis, where hair loss occurs over multiple areas;
Ophiasis Alopecia areata, where there is a sudden thinning of hair as opposed to hair loss in patches
Alopecia areata barbae, where the hair loss is limited to the beard;
Alopecia areata totallis, where there is total hair loss on the scalp; and
Alopeica areata universalis, where all body hair is lost, including pubic hair.

There are no known cures for alopecia. Treatment options typically include corticosteroids such as clobetasol or fluocinonide, minoxidil, immunosuppressants such as cyclosporine, and diphenylcyclopropenone. Wigs or hairpieces can be used to cover up the bald areas, although some patients may find these uncomfortable to wear.

Vitiligo and Alopecia areata are not life-threatening conditions and are not contagious. However, the symptoms of both diseases are highly visible and can therefore also have an adverse effect on the emotional and mental health of patients suffering from either or both diseases.

In this respect, studies have shown that vitiligo and alopecia tend to occur together more commonly in patients compared to pure chance. In addition, both diseases feature a patchy distribution and are minimally symptomatic, compared to other more inflammatory skin conditions like atopic dermatisis or psoriasis. The similarities between the two autoimmune skin diseases further extend beyond shared clinical symptoms-they also share similarities in pathogenesis. For example, increase reactive oxygen species (ROS) and high cellular stress levels are thought to act as the initiating trigger for the innate immune system in both conditions. In addition, studies in mouse models have implicated an IFN-γ-driven immune response and cytotoxic CD8+ T cells as the main pathogenic factors in both diseases. See review by Rork et al, Curr Opin Pediatr 2016 August; 28 (4): 463-469. In view of the implication of the IFN-γ signalling pathway in both diseases, ruxolitinib (an inhibitor that targets JAK, a downstream effector of IFN-γ signalling) is currently undergoing clinical trials for both vitiligo and alopecia.

Hence, whilst not wishing to be bound by theory, the present inventors believe that farudostat has the potential to treat both vitiligo and alopecia by virtue of their various similarities, in particular the crucial role of cytotoxic CD8+ T cells in both diseases.

“Inadequate control” as employed herein refers to where standard of care medication fails to lessen or control symptoms, in particular where the patient's quality of life is adversely affected.

“Defined endpoint” as employed herein refers to clinically defined point, for example remission or stable disease.

PMEL Melanocyte protein PMEL also known as premelanosome protein (PMEL) or silver locus protein homolog (SILV) is a protein that in humans is encoded by the PMEL gene. Its gene product may be referred to as PMEL, silver, ME20, gp100 or Pmel17. PMEL is a 100 kDa type I transmembrane glycoprotein that is expressed primarily in pigment cells of the skin and eye. The human protein has the UniProt no. P40967.

### Formulations

A DHODH inhibitor is a moiety (such as a compound) that inhibits, for example reduces or blocks the activity of a DHODH enzyme (see background for definition thereof).

In one embodiment the DHODH inhibitor is provided as a pharmaceutical formulation or pharmaceutical composition.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes.

In one embodiment the pharmaceutical formulation is for oral administration, for example formulated as tablets, pills, dragees, capsules, liquids, gels, syrups, slurries and suspensions, for ingestion by the patient.

Excipients may include lactose, dextrin, glucose, sucrose, sorbitol, starch, sugars, sugar alcohols and cellulose.

Other suitable forms for administration include parenteral administration, for example injection or infusion, such as bolus injection or continuous infusion.

Where the product is for injection or infusion, it may take the form of a suspension, solution or emulsion in an oily or aqueous vehicle and it may contain excipients, such as a suspending agent, preservative, stabilising and/or dispersing agents. Alternatively, the molecule may be in dry form, for reconstitution before use with an appropriate sterile liquid. Pharmaceutically acceptable carriers in therapeutic compositions may additionally contain liquids such as water, saline, glycerol and ethanol. Additionally, auxiliary substances, such as wetting agent, emulsifying agents, lubricant or pH buffering substances, may be present in such compositions.

A thorough discussion of pharmaceutically acceptable carriers is available in Remington's Pharmaceutical Sciences (Mack Publishing Company, N.J. 1991).

### Treatment

Treatment as employed herein refers to where the patient has a disease or disorder, for example autoimmune disease (in particular one disclosed herein) and the medicament according to the present disclosure is administered to stabilise the disease, delay the disease, ameliorate the disease, send the disease into remission, maintain the disease in remission or cure the disease. Treating as employed herein includes administration of a medicament according to the present disclosure for treatment or prophylaxis.

Treatment or therapy may be employed prophylactically.

Therapeutically effective amount as employed herein is an amount in the range which generates a desirable physiological effect, whilst minimising side effects.

Disease modifying therapy as employed herein refers to therapy that allows the immune system to reset itself and rebalance, thereby performing more normally after treatment.

The DHODH inhibitor of the disclosure or formulation comprising the same may be administered at a dose in the range of 1 mg to 400 mg per day, such as 10 mg to 400 mg per day, 50 mg to 400 mg per day, 100 mg to 400 mg per day, 150 mg to 400 mg per day, 200 mg to 400 mg per day, 250 mg to 400 mg per day, 300 mg to 400 mg per day, or 350 mg to 400 mg per day.

In particular, a dose in the range of 100 mg to 400 mg per day is administered.

Thus, the daily dose may be for example 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg.

In one embodiment each dose is 1 to 150 mg/kg (for example 5 to 150 mg/kg), such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 8-, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132 133, 134, 135, 136, 137, 139, 140, 141, 142, 143, 144, 145, 146, 147, 148, 149 or 150 mg/kg.

In one embodiment the treatment (dose) is administered daily, for example once or twice daily.

In one embodiment the treatment (dose) is administered once daily.

In one embodiment the dose for alopecia is 100 mg to 400 mg once or twice a day, for example 200 mg to 300 mg

In one embodiment the dose for vitiligo is 100 mg to 400 mg once or twice a day, for example 100 to 200 mg.

In one embodiment farudostat is employed in a maintenance therapy, for example at a low dose. Maintenance therapy as employed herein refers to continuous therapy to make the disease stable or to keep the disease in remission, for example where the dose administered is low and in particular frequent. A dose of 100 to 200 mg for example given once or twice a day may be used as a maintenance therapy.

In one embodiment farudostat is administered orally, for example as a tablet or capsule or caplet.

Co-morbidity as employed herein refers to where the patient is suffering from a second or underlying health condition.

Combination therapy (comprising further therapy) as employed herein wherein two or more treatment regimens are employed, in particularly employed concomitantly. The treatments may be separate formulations or co-formulated. They may be administered at the same time or different times. However, the pharmacological effect of the treatments will co-exist in the patient.

Further therapy as employed herein refers to a therapy in addition to the DHODH inhibitor.

Such a further therapy may be an anti-inflammatory agent, which includes but is not limited to, a non-steroidal anti-inflammatory agent (NSAID), a disease modifying anti-rheumatic drug (DMARD), a statin (including HMG-COA reductase inhibitors such as simvastatin), a biological agent (biologicals), a steroid, an immunosuppressive agent, a salicylate and/or a microbicidal agent.

Non-steroidal anti-inflammatory agents include anti-metabolite agents (such as methotrexate) and anti-inflammatory gold agents (including gold sodium thiomalate, aurothiomalate or gold salts, such as auranofin). Biologicals include anti-TNF agents (including adalimumab, etanercept, infliximab, anti-IL-1 reagents, anti-IL-6 reagents, anti-CD20 agents, anti-B cell reagents (such as rituximab), anti-T cell reagents (anti-CD4 antibodies), anti-IL-15 reagents, anti-CLTA4 reagents, anti-RAGE reagents), antibodies, soluble receptors, receptor binding proteins, cytokine binding proteins, mutant proteins with altered or attenuated functions, RNAi, polynucleotide aptamers, antisense oligonucleotides or omega 3 fatty acids. Steroids (also known as corticosteroids) include cortisone, prednisolone or dexamethasone may also be employed in a combination therapy.

Immunosuppressive agents for use in a combination therapy according to the present disclosure include cyclosporin, FK506, rapamycin, mycophenolic acid. Salicylates for use in said combination therapy include aspirin, sodium salicylate, choline salicylate and magnesium salicylate. Microbicidal agents include quinine and chloroquine.

Anti-inflammatory as employed herein refers to a moiety that reduces inflammation, for example a non-steroidal anti-inflammatory, steroids and the like.

In one embodiment, the combination therapy comprises an anti-CD20 agent or a biosimilar thereof, for example Rituxan (rituximab), a Rituximab biosimilar, Gazyva, Kesimpta, Ocrevus (ocrelizumab), Ruxience, Truxima, Zevalin, Arzerra, AcellBia, HLX01, Reditux, Ritucad or Zytux.

In one embodiment, the combination therapy comprises a treatment independently from corticosteroids example oral prednisone and intravenous (for selected methylprednisolone), plasma exchange (plasmapheresis), interferon beta medications, glatiramer acetate, fingolimod, dimethyl fumarate, diroximel fumarate, teriflunomide, siponimod, cladribine, ocrelizumab, natalizumab and alemtuzumab.

In one embodiment, the combination therapy comprises a treatment to ease or reduce the symptoms of multiple sclerosis, for example a muscle relaxant (such as baclofen, tizanidine and cyclobenzaprine), a medication to reduce fatigue (such as amantadine, modafinil, methylphenidate, or a medication to increase walking speed (such as dalfampridine).

In one embodiment the combination therapy comprises cannabis or a derivative thereof, for example cannabis oil.

In one embodiment the combination therapy comprises a second DHODH inhibitor. In one embodiment, the further therapy comprises teriflunomide. In one embodiment the further therapy comprises vidofludimus. In one embodiment the combination therapy does not comprise a second DHODH inhibitor. In embodiment the combination therapy does not comprise teriflunomide and/or vidofludimus.

In one embodiment the combination therapy comprises a disease modifying therapy, for example selected from alemtuzumab, avonex, betaferon, cladribine, daclizumab, dimethyl fumerate, extavia, fingolimod, glatiramer acetate, natalizumab, ocrelizumab, plegridy, rebif, siponimod and combinations of two or more of the same.

In one embodiment the further therapy comprises an inhibitor of interferon-gamma (IFN-γ) signalling, such as an IFN-γ inhibitor or a JAK inhibitor. In one embodiment the further therapy comprises an IFN-γ inhibitor. In one embodiment, the further therapy comprises an anti-IFN-γ antibody, such as an anti-IFN-γ receptor antibody. In one embodiment, the IFN-γ inhibitor is selected from the group comprising dexamethasone, mesopram, GIT27 and rocaglamide. In one embodiment, the further therapy comprises a JAK inhibitor, for example an inhibitor of JAK1, JAK2, JAK2 and/or TYK2. In one embodiment, the further therapy comprises an IFN-γ inhibitor. In one embodiment, the further therapy comprises a JAK inhibitor, such as an anti-JAK antibody. In one embodiment, the JAK inhibitor is selected from the group comprising ruxolitinib, tofacitinib, oclacitinib, baricitinib, peficitinib, fedratinib, updadacitinib, filgotinib, delgocitinib, abrocitinib, cerdulatinib, gandotinib, lestaurtinib, momelotinib, pacritinib, deucravacitinib, cucurbitacin, and CHZ868. In one embodiment, the JAK inhibitor is selected from the group comprising ruxolitinib, tofacitinib, oclacitinib, baricitinib, peficitinib, fedratinib, updadacitinib, filgotinib, delgocitinib and abrocitinib.

In one embodiment, the combination therapy comprises a Bruton's Tyrosine Kinase (BTK) inhibitor, for example Ibrutinib, Acalabrutinib, Zanubrutinib, Evobrutinib, ABBV-105, Fenebrutinib, GS-4059, Spebrutinib and/or HM71224.

In one embodiment, the further therapy comprises glatiramer acetate. In one embodiment, the further therapy comprises natalizumab. In one embodiment, the further therapy comprises mitoxantrone. In one embodiment, the further therapy comprises fingolimod. In one embodiment the further therapy comprises Siponimod. In one embodiment, the further therapy comprises dimethyl fumarate. In one embodiment, the further therapy comprises alemtuzumab. In one embodiment, the further therapy comprises cyclophosphamide. In one embodiment, the further therapy comprises cladribine. In one embodiment, the further therapy comprises ocrelizumab. In one embodiment, the further therapy comprises dimethyl fumarate. In one embodiment, the further therapy comprises daclizumab. In on embodiment, the further therapy comprises azathioprine. In one embodiment, the further therapy comprises methotrexate. In an alternative embodiment, the further therapy does not comprise methotrexate. In one embodiment, the further therapy comprises lacquinimod.

Comprising in the context of the present specification is intended to mean “including”.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The background contains technical information and may be used as basis for amendment.

The invention will now be described with reference to the following examples, which are merely illustrative and should not be construed as limiting the scope of the present invention.

### EXAMPLES

### Example 1—Study to Assess Suitability of Farudostat for Treatment of Vitiligo

The suitability of farudostat for the treatment of vitiligo was assessed using a mouse model which closely resembles vitiligo in humans.

### Mouse Model

Using transgenic Krt14-KitL* (SCF) mice, which retain melanocytes in the skin, vitiligo was induced in the mice using recombinant vaccinia virus expressing hgp100 or BMDCs pulsed with hgp100 (Harris J E, et al. JID 2012; Riding R L; et al. Current Protoc Immunol 2018; Riding, R. L., et al. PCMR 2020).

The models are driven by a population of antigen-specific autoreactive CD8+ T cells that recognize a melanocyte-specific protein (PMEL), which mirrors human vitiligo pathogenesis. The adoptively transferred cells induce disease and can be tracked after transfer at any site (skin, blood, spleen, lymph nodes, etc.) by congenic or fluorescent reporter proteins. (Van den Boorn, J G, et al. JID 2009; Harris J E, et al. JID 2012)

The following treatment groups were tested:
1. Vehicle 0.5% methylcellulose (negative control)
2. 75 mg/kg/day farudostat
3. 150 mg/kg/day farudostat
4. 250 μg anti-IFNγ twice a week (positive control)

Vehicle and farudostat treatments were administered via oral gavage. Anti-IFNγ treatments were administered via intraperitoneal (IP) injection.

The treatment was carried out over a period of 6 weeks, with 5 days of treatment a week, i.e. 30 days of actual treatment.

### Vitiligo Scoring

Disease severity was scored by a trained investigator, who subjectively scored each SCF mouse by estimating the percent depigmentation at each of the 4 skin sites—the nose, ears, rear footpads and tail. Both the left and right ears and the left and right rear footpads are estimated together and evaluated as a single site.

Table 1 below shows the score assigned per site based on the estimated % depigmentation.

TABLE 1
Vitiligo Scoring system in mouse model

| SCORE | 0 | 1 | 2 | 3 | 4 | 5 |
|---|---|---|---|---|---|---|
| TAIL | 0 | <10% | 10-25% | >25-75% | >75-99% | 100% |
| EARS | 0 | <10% | 10-25% | >25-75% | >75-99% | 100% |
| NOSE | 0 | <10% | 10-25% | >25-75% | >75-99% | 100% |
| FOOTPADS | 0 | <10% | 10-25% | >25-75% | >75-99% | 100% |

Thus, the highest score each mouse can achieve is 20.

Vitiligo scoring was performed at the end of the experiment.

### Flow Cytometry

Tissue samples from all animals were obtained at the end of the experiment, according to previously published protocols (Riding R L, et al., Current Protocol Immun 2019). Briefly, the entire tail, split into dermis and epidermis, spleen and skin-draining lymph nodes, were processed mechanically and/or chemically, prepared and stained for flow cytometry.

A basic flow panel was used to stain for the following markers: Live/Dead, CD45, CD8, CD3 and Thy1.1, which is the PMEL T cell marker of the adoptively transferred cells. Hence, the measurement of PMEL T cell counts provides an indication of depigmentation, which is a hallmark symptom of vitiligo. Flow cytometry data analysis was conducted using FlowJo software.

### Results

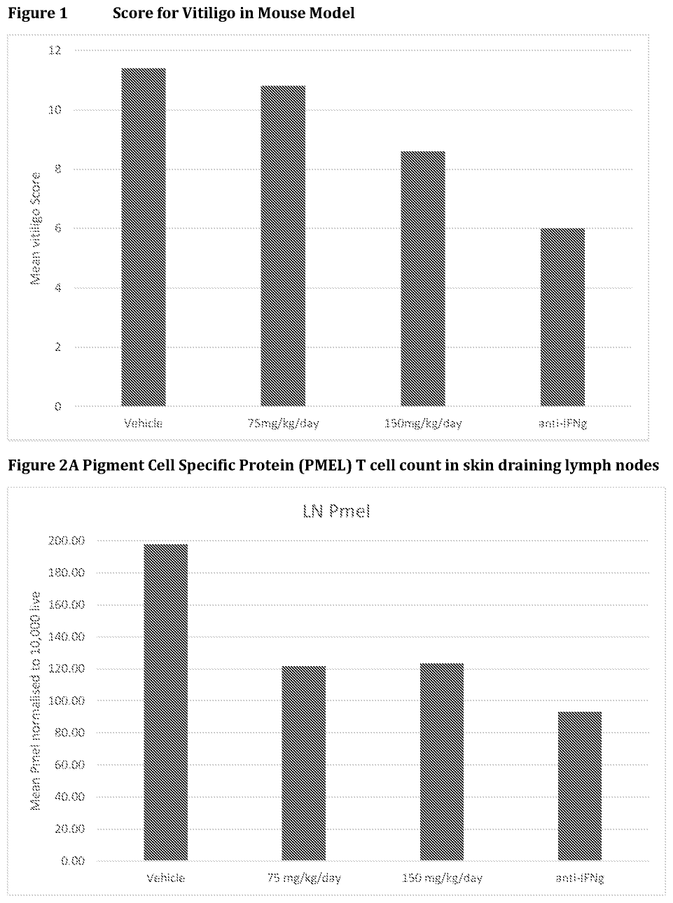

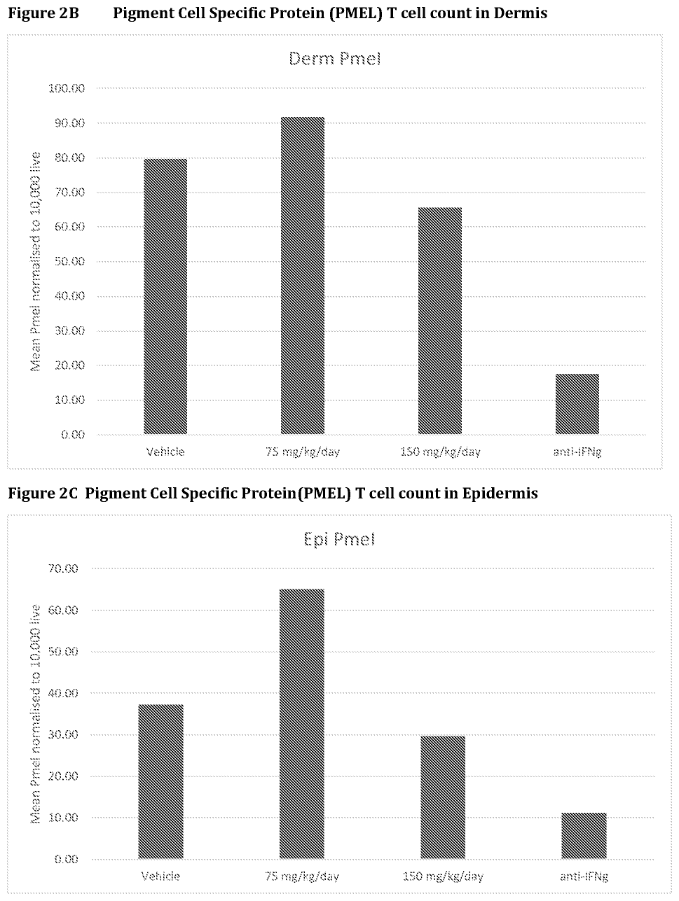

The results of the study are shown in FIG. 1 and FIGS. 2A to 2D.

FIG. 1 shows the final mean vitiligo score for each treatment group. As can be seen, the administration of farudostat at both 75 mg/kg/day and 150 mg/kg/day resulted in an improvement in vitiligo score compared to the vehicle treated control group. The 150 mg/kg/day group had a greater improvement in vitiligo score compared to the 75 mg/kg/day group. FIGS. 2A to 2C show the mean PMEL T cell counts for skin draining lymph nodes (FIG. 2A) dermis (FIG. 2B), epidermis (FIG. 2C) and spleen (FIG. 2D) for each treatment group, assessed using flow cytometry.

The graphs indicate a general trend of farudostat treatment resulting in a reduction in mean Pmel T cell count, i.e. a reduction in depigmentation for dermis and epidermis. Treatment with 150 mg/kg/day farudostat typically resulted in a greater reduction in mean PMEL count compared to 75 mg/kg/day. Interesting, farudostat treatment produced a reduction in mean PMEL count in the spleen that was even greater than that observed for anti-IFNγ (see FIG. 2D).

In conclusion, the results demonstrate that farudostat was able to improve the symptoms of vitiligo in the mouse model, suggesting farudostat's strong potential as a treatment for vitiligo.

### Example 2—Study to Assess Suitability of Farudostat for Treatment of Alopecia

The suitability of farudostat for the treatment of alopecia was assessed using a mouse model which closely resembles alopecia in humans.

### Mouse Model

The C3H/HeJ mouse strain is known to spontaneously develop alopecia (AA) at a low frequency over a prolonged time frame. Transplantation of AA-affected skin from a C3H/HeJ mouse to unaffected C3H/HeJ recipients will induce the development of AA in nearly 100% of recipients over 4-8 weeks.

Accordingly, to produce the AA mouse model, AA-affected C3H/HeJ skin was grafted onto groups of unaffected C3H/HeJ mice.

The following treatment groups (n=10 per treatment group) were tested:
1. Vehicle 0.5% methylcellulose (negative control)
2. 150 mg/kg/day farudostat
3. 300 mg/kg/day farudostat

Treatments were administered once daily by oral gavage starting 2 weeks after engraftment and concluded after 12 weeks (84 days) of treatment.

### Photographs

Photographs were taken at baseline, and every 4 weeks until the end of study

### Hair Growth Index

Ventral hair growth was scored based on the photographs. Any mouse that had >98% hair coverage was classified as ‘no hair loss’.

### Flow Cytometric Analysis

Flow cytometry will be performed on affected or representative skin and the skin-draining lymph nodes. Cell extracted from the skin or skin-draining lymph nodes will be stained for CD3, CD8, CD4, NKG2D, CD45, and Live/Dead.

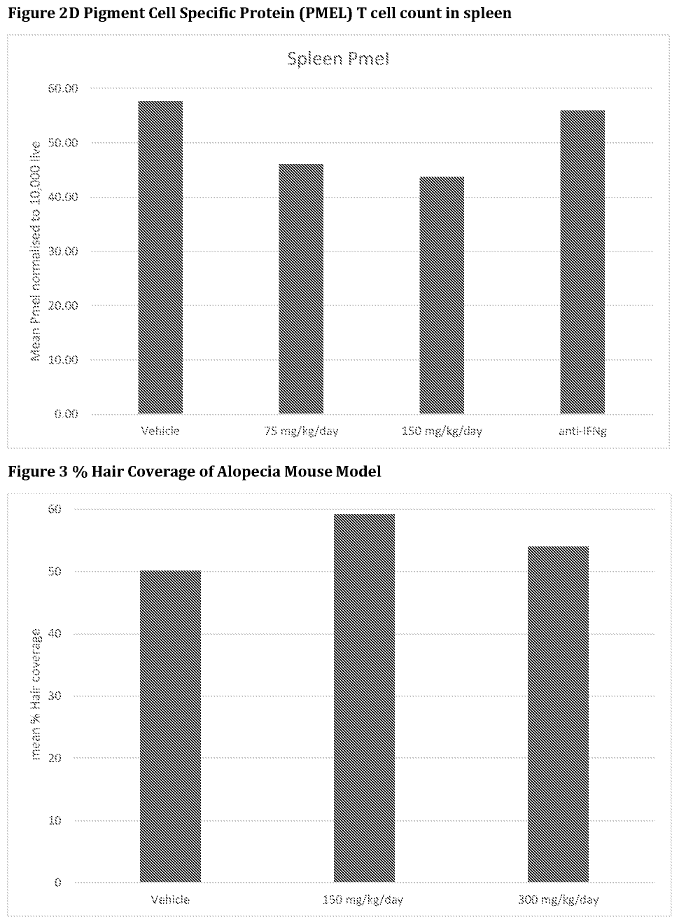

FIG. 3 shows a graph of the mean % hair coverage (i.e. the % body surface area that had hair on a per-mouse basis) at the week 8 time point (i.e. 10 weeks after engraftment). The data suggests that the mice treated with farudostat had a slight improvement in % hair coverage compared to the mice treated with vehicle.

In summary, the interim 8-week data suggests that farudostat can reduce hair loss and may potentially be suitable for treating Alopecia areata.

1.-16. (canceled)

17. A method of treating an autoimmune skin disease comprising administering a therapeutically effective amount of A DHODH inhibitor 2-(3,5-difluoro-3′methoxybiphenyl-4-ylamino) nicotinic acid or a pharmaceutically acceptable salt thereof.

18. The method according to claim 1, wherein the autoimmune skin disease is selected from the group comprising: vitiligo, alopecia, atopic dermatitis, Behcet's disease, dermatitis herpetiformis, dermatomyositis, lichen planus, linear IgA disease, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, and vesiculobullous dermatosis.

19. The method according to claim 1, wherein the autoimmune disease is not one or more of the following: atopic dermatitis, Behcet's disease, dermatitis herpetiformis, dermatomyositis, lichen planus, linear IgA dermatosis, lupus of the skin, morphea/scleroderma, ocular cicatrical pemphigoid, pemphigoid such as bullous pemphigoid or pemphigoid gestationis, pemphigus (such as pemphigus vulgaris, pemphigus vulgaris, pemphigus foliaceaous, pemphigus erythematosus, IgA pemphigus, pemphigus vegetans, or paraneoplastic pemphigus), vasculitis, epidermolysis bullosa acquisita, psoriasis, or vesiculobullous dermatosis.

20. The method according to claim 1, wherein the autoimmune disease is autoimmune skin disease is selected from the group comprising vitiligo and alopecia.

21. The method according to claim 1, wherein the autoimmune skin disease is vitiligo, such as segmented or non-segmented vitiligo.

22. The method according to claim 1, wherein the autoimmune disease is alopecia, such as diffuse Alopecia areata, Alopecia areata monolocularis, ophiasis Alopecia areata, alopecia areata barbae, Alopecia areata totallis or Alopecia areata universalis.

23. The method according to claim 1, wherein the autoimmune disease is characterized by aberrant T cell and/or B cell activation.

24. The method according to claim 1, wherein the autoimmune disease is severe.

25. The method according to claim 1, wherein the DHODH inhibitor is employed as monotherapy.

26. The method according to claim 1, wherein the DHODH inhibitor is employed in a combination therapy.

27. The method according to claim 1, wherein the DHODH inhibitor is employed in a combination therapy, and the combination therapy comprises a treatment independently selected from: a corticosteroid, an immunosuppressant, and/or a purine synthesis inhibitor (such as azathioprine).

28. The method according to claim 1, wherein the DHODH inhibitor is employed in a combination therapy, and the combination therapy comprises phototherapy.

29. The method according to claim 1, wherein the DHODH inhibitor is administered at a dose in the range of 1 mg to 400 mg per day, for example 100 mg to 400 mg per day, such as 100, 200, 300 or 400 mg.

30. The method according to claim 1, wherein the DHODH inhibitor is administered daily, for example once daily.
